(19) United States
(12) Patent Application Publication
(10) Pub. No.: US 2024/0201128 A1
(43) Pub. Date: Jun. 20, 2024

(54) METHOD FOR CALIBRATING AN AMPEROMETRIC SENSOR AT A STATIONARY MEASURING POINT AND DEVICE FOR ON-SITE CALIBRATION OF THE AMPEROMETRIC SENSOR

(71) Applicant: Endress+Hauser Conducta GmbH+Co. KG, Gerlingen (DE)

(72) Inventors: Holger Lippert, Leisnig (DE); Jonathan Weinbrenner, Schönaich (DE); Erik Hennings, Freiberg (DE); Nora Schubert, Meißen (DE)

(21) Appl. No.: 18/543,208

(22) Filed: Dec. 18, 2023

(30) Foreign Application Priority Data
Dec. 20, 2022 (DE) 10 2022 134 218.2

(51) Int. Cl. G01N 27/416; G01N 33/18

(52) U.S. Cl. CPC G01N 27/4163; G01N 33/18

(57) ABSTRACT

A method for calibrating an amperometric sensor at a stationary measuring point, comprises the following steps: providing a mobile calibration device comprising a stabilized calibration fluid at the stationary measuring point; activating the stabilized calibration fluid and providing an activated calibration fluid; calibrating the amperometric sensor by means of the activated calibration fluid; and a mobile calibration device.

### CROSS-REFERENCE TO RELATED APPLICATION

The present application is related to and claims the priority benefit of German Patent Application No. 10 2022 134 218.2, filed on Dec. 20, 2022, the entire contents of which are incorporated herein by reference.

### TECHNICAL FIELD

The present disclosure relates to a method and to a device for the in-situ calibration of an amperometric sensor at a measuring point.

### BACKGROUND

Amperometric disinfection sensors are used in various applications with different organic and inorganic loads, for example in the field of drinking water and bathing water treatment, treatment in seawater desalination plants, treatment of process water, etc.

The sensors are usually calibrated by determining the concentration of disinfectant in a sample of the process water by means of a photometric reference measurement (DPD), and the sensor is calibrated to this value. The reference method and the sensor are usually cross-sensitive to various disinfectants, or can be disturbed by interfering ions, particles or other influencing factors in the water. Due to a lack of stable calibration solutions, the photometric reference method is also used for external calibration of sensors outside the process.

As explained previously, customers must thus use photometric reference measurements to calibrate and maintain disinfection sensors, because there are currently no stable calibration solutions for calibrating amperometric disinfection sensors.

The photometric reference measurements are highly error and user dependent. Errors are not uncommon in the range of 10-20% depending on the concentration value.

In applications with very low concentration values, for example in drinking water treatment and distribution, very low concentrations of disinfectant are used and the error incurred in the photometric reference method used to calibrate the sensors is particularly large here.

Increasingly, mixtures of disinfectants, for example free chlorine and chlorine dioxide or mixtures with other cleaning agents and biocides, are also used in the customers' process, to which sensors and reference measurement have varying degrees of cross sensitivity, making in-situ calibration directly in the process water of the customer very difficult.

In applications that are heavily loaded with consuming biofilms, organic material or inorganic reducing agents, calibration in the process is also difficult and prone to error. Applications having a high turbidity content likewise imply a high level of susceptibility to errors in photometric reference measurements.

A further application results from the fact that the sensors and fittings must be dismantled partially or completely in order to clean them. Cleaning is generally done manually using suitable solutions, rinsing, and tools such as brushes.

It is not possible to sample the measuring medium, for example water, for the purpose of determining measurement data, because the composition determined in the laboratory, in particular with regard to the concentration of disinfectant is contains, does not correspond to the composition at the time of sampling.

In summary, the photometric reference method is cross-sensitive, error-prone and highly user-dependent. In the case of mixtures of different disinfectants, in-process calibration is thus not possible.

It is not possible to subject water samples from the process to costly laboratory analyses either. Because the disinfectants are not stable and degrade over time, the sample changes continuously immediately after collection. The results of the laboratory analyses are thus already subject to error due to the degradation of the disinfectant between sampling and analysis. In the aforementioned example, the sampling time and the calibration time are also too far apart from one another, and the measuring water may have changed during this time. This also introduces additional error potential.

### SUMMARY

Proceeding from the aforementioned prior art, the present disclosure therefore begins with the object of reducing sources of error in the calibration of the sensor.

The present disclosure achieves this object by a method having the features of claim 1 and by a calibration device having the features of claim 12.

A method according to the disclosure for calibrating an amperometric sensor at a stationary measuring point has at least the following steps:
A: Providing a mobile calibration device comprising a stabilized calibration fluid at the stationary measuring point.

A mobile calibration device is, for example, a calibration case or the like. The calibration device can, for example, weigh less than 30 kg, preferably less than 20 kg.

The stabilized calibration fluid is a fluid that is protected from decomposition.

The measuring point is a measuring point in process measurement technology. In this application, the measuring point is a fixed technical term.

The provision at the stationary measuring point can only include a position of the calibration device in the vicinity of the measuring point. In a particularly preferred embodiment, in which the installation position of the amperometric sensor remains unchanged, the activated calibration fluid is introduced into the measuring point in such a way that it is introduced, for example, into a sensor fitting on a process line or a process container and thereby comes into contact with the amperometric sensor without removing said sensor.
B: Activating the stabilized calibration fluid and providing an activated calibration fluid;

In a subsequent step, the stabilized calibration fluid is activated. For example, stabilization can be ensured at a certain pH with a stabilizer, for example sodium pentaphosphate. When the pH is changed, for example by adding an acid or base, conversion to a species that can be detected by the amperometric sensor can occur.

This calibration solution provided immediately prior to calibration allows for accurate calibration of the amperometric sensor. Because said calibration solution is subject to a time conversion, its supply only makes sense in the context of in-situ calibration. In particular, the sensor can remain in its measurement position at this position.
C: Calibrating the amperometric sensor by means of the activated calibration fluid

Finally, calibration of the amperometric sensor is carried out. A number of variants are provided for this purpose, which can also advantageously be combined with one another for redundant checking.

Advantageous embodiments of the method according to present disclosure form the subject matter of the dependent claims.

The amperometric sensor can be fixed at the measuring point of a process line or a process container when method steps A-C are carried out. A change in the sensor position at the measuring point can lead to erroneous measurements and in particular to incorrect assessment in comparison with long-term measurement protocols.

In special situations, however, the advantage of a calibration process outside the measuring point outweighs the disadvantages. This is the case, for example, with heavy soiling, in particular the formation of a biofilm on the sensor surface or the like. Intensive mechanical and/or chemical cleaning of the amperometric sensor makes sense here.

In practical terms, the amperometric sensor can then be provided in a holding device of the calibration device when method step C is carried out. This implies prior removal of the amperometric sensor from the measuring point and a reinstallation at the measuring point after calibration.

In addition, a particular advantage is provided if the process water or the process fluid contains different disinfectants to which the amperometric sensor is cross-sensitive. The sensor can thus be calibrated with greater accuracy by applying the present disclosure with an activated calibration fluid that contains a single disinfectant, preferably preferably a halogen compound and/or an oxidizing agent, particularly preferably free chlorine, chlorine dioxide, free bromine or hydrogen peroxide.

After step B, the activated calibration fluid can also be supplied to the measuring point so that the calibration process can take place without removal of the amperometric sensor.

As described above, activation can take place by the addition of a pH-changing reactant. Such pH-dependent reactions generally proceed at a comparatively high reaction rate so that little time elapses between the start of the addition and the complete conversion to the free measurable species.

It is advantageous if the activation, i.e., the start of the addition of the reactant, and the calibration of the amperometric sensor take place within less than 10 minutes. The calibration process within this time must therefore take place at the measuring point, because no longer distances with respect to the measuring distance can be covered within the time window.

The method can comprise, preferably at least prior to the calibration process in step C, supplying cleaning liquid to the measuring point. The measuring point is thereby conditioned. Ideally, organic or inorganic deposits, such as algae, lime or the like, are removed from the measuring point. The cleaning agent can, for example, contain an algicide and/or have an acidic pH for breaking down lime. The cleaning liquid can optionally be heated by a temperature control device to increase effectiveness. Corresponding flow heaters are available in a miniaturized design and can be arranged in a supply line between the calibration device and the measuring point.

The calibration process can furthermore comprise detecting an actual value by means of the amperometric sensor and comparing a target/actual value, wherein the target value is determined from a predefined concentration of a compound in the stabilized calibration fluid, which is converted into a species recordable by the amperometric sensor during the activation process in step B. This variant is accompanied by low apparatus expenditure, which leads to a reduction in the size of the calibration device and/or to provision of a larger amount of fluid in the calibration device.

After calibration, the amperometric sensor can either be re-inserted into the measuring point or the fluid lines, in particular at least one supply line, between the calibration device and the measuring point can be removed. As a result, the calibration device is available for any other measuring point.

The calibration process in step C can comprise measuring the calibration fluid by means of the amperometric sensor and measuring the calibration fluid by means of a reference measurement, preferably an amperometric measurement, and/or determining the turbidity content and/or a photometric measurement, optionally supplemented by a pH measurement.

A mobile calibration device according to the present disclosure for carrying out the method according to the present disclosure is designed as a portable device. The calibration device can preferably have a case-shaped housing having an upper and a lower case shell.

In a minimal configuration, the calibration device can have a storage tank containing a stabilized calibration fluid and a pump for transporting the calibration fluid to the measuring point or to an internal holding device for the amperometric sensor.

In principle, the aforementioned method according to the present disclosure can also have no storage tank inside the calibration device, but rather the corresponding solutions can be sucked out of storage containers that are carried along, for example with a capacity of 10 liters. In this respect, the calibration device according to the present disclosure is a special embodiment of the calibration device of the method according to the present disclosure.

The reactant for releasing the detectable species can be added to the storage tank containing the stabilized calibration fluid in the simplest manner as a tablet or by other means. Therefore, a storage tank for the reactant does not necessarily have to be part of the calibration device.

In the aforementioned example, however, filling of the storage tank with the calibration fluid is intended for a single calibration. The fluid in the storage tank must then be completely replaced.

It is also possible to carry out a plurality of calibrations using the calibration device without replacing the calibration fluid. For this purpose, the calibration device has a storage container for a reactant for activating the calibration fluid and preferably a metering device for metering the reactant into the storage tank containing the stabilized calibration fluid or preferably into a mixing and reaction tank.

The mixing and reaction tank is recommended in particular for multiple use. The metering device can be a fluid metering system or optionally only a solid metering system, for example an access port for supplying a tablet or another form, for example powder or the like.

To carry out the calibration process, a control and/or evaluation unit can be provided, which can be part of the calibration device or can be an external device, for example a mobile terminal, for example having a corresponding calibration application. The latter makes it possible to reduce the size of the calibration device.

The mobile calibration device can advantageously have an integrated power supply, preferably a battery or another power storage device, so that an autonomous mode of operation is possible.

The calibration device can additionally advantageously have a flow-through fitting having a sensor element, for example a photometer or an amperometric reference sensor, wherein the sensor element is preferably fluidically connected to an inlet that can be connected to the measuring point or is connected to the holding device of the calibration device for holding the amperometric sensor. In the first case, the calibration solution can be returned to the calibration device for the execution of a reference measurement after passing through the measuring point. Interference factors, such as impurities in the calibration fluid, can thereby be compensated for. Alternatively, the freshly prepared calibration fluid can also be supplied for the reference measurement first and then to the measuring point.

The present disclosure further comprises the use of the calibration device for calibrating a sensor for determining the content of a disinfectant in a process fluid, in particular process water, wherein the process fluid contains a mixture of a plurality of disinfectants.

Because the process fluid contains this mixture, step A, i.e., providing the calibration device at the mobile measuring point, can also comprise emptying the process line or the process container, wherein the process fluid to be emptied contains the mixture of a plurality of disinfectants.

### BRIEF DESCRIPTION OF THE DRAWINGS

In the following, the subject matter of the present disclosure is explained in detail using an exemplary embodiment and with the aid of accompanying figures. In the figures:

FIG. 1 shows a process diagram of a method according to the present disclosure with a calibration device according to the present disclosure; and

FIG. 2 shows a schematic view of a calibration device according to the present disclosure in the process; and

FIG. 3 shows a detailed view of the calibration device of FIG. 2.

### DETAILED DESCRIPTION

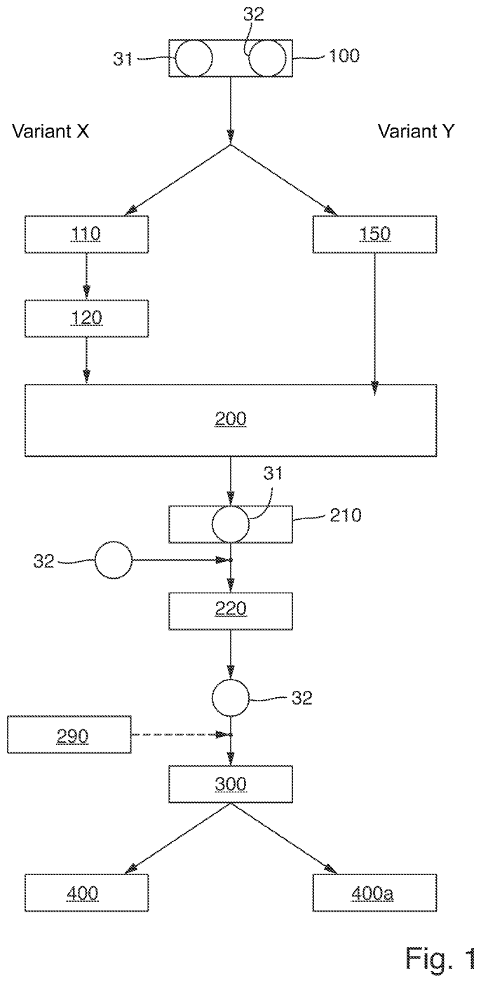

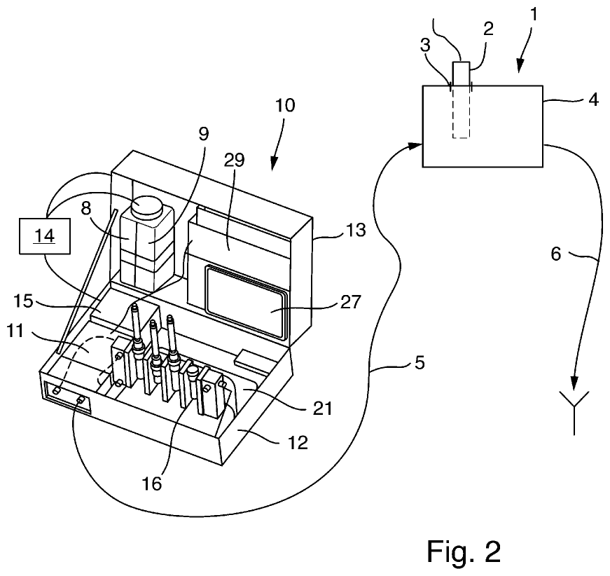

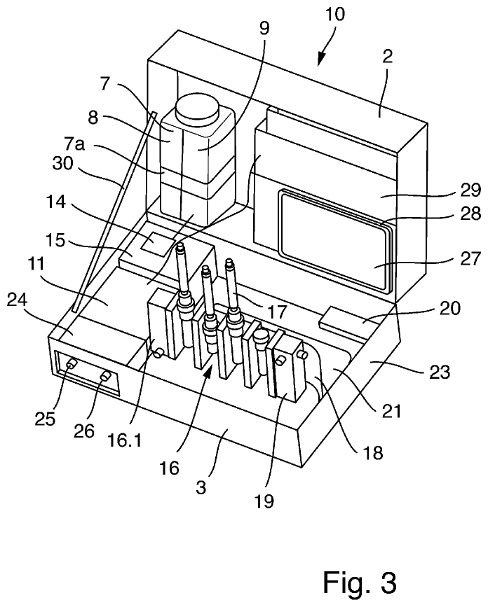

FIG. 1 shows the course of the method for two variants of the method according to the present disclosure. The method is carried out by means of a mobile calibration device 10, which is shown in detail in FIGS. 2 and 3.

The method relates to the on-site calibration of a sensor at a stationary measuring point. In the following, a measuring point refers to a physical facility at which a physical and/or chemical parameter of a measuring medium is recorded in relation to a measuring point over a defined period of time.

In the present case, the measuring medium preferably refers to a measuring medium containing disinfectant, for example for disinfecting water. Using disinfectant, for example H₂O₂, free chlorine, chlorine dioxide or even bromine, is usually accompanied by decomposition of the disinfectant over a contact time.

The sensor is an amperometric sensor 2 that is fixed in a process line 4 or a process container via a holding device 3. This is the aforementioned measuring point 1. Amperometric sensors are known per se. The measuring principle of such a sensor for determining free chlorine is briefly explained below.

Free chlorine is determined using hypochlorous acid according to the amperometric measuring principle. The hypochlorous acid (HOCl) contained in the medium diffuses through a sensor membrane and is reduced to chloride ions (Cl−) at a downstream cathode of the sensor. However, other variations of chlorine determination, for example using a platinum cathode, are also possible. Furthermore, the sensor has a silver anode. Silver is oxidized to silver chloride at this silver anode. The electron release at the cathode and the electron uptake at the silver anode results in a current flow that is proportional to the concentration of free chlorine in the medium under constant conditions. The electrodes are located in an electrolyte that is separated from the medium by the aforementioned sensor membrane. The sensor membrane prevents the electrolyte from flowing out and protects it from the ingress of foreign substances. The concentration of the hypochlorous acid depends on the pH in this case. This dependence can be compensated for via an additional pH measurement. The measuring transducer calculates the concentration of free chlorine in mg/l (ppm) measured variable from the current signal.

Amperometric sensors are not only suitable for the determination of chlorine, but also bromine, chlorine dioxide, H₂O₂ and other disinfectants. Typically, such an amperometric sensor is calibrated by what is known as an optical reference measurement, also called DPD. The optical reference measurement is carried out by means of a photometer and with the aid of an indicator reagent, wherein DPD is the abbreviation for the indicator reagent (N,N-diethyl-p-phenylenediamine), which is slightly pink in the presence of free chlorine. This change in color can be measured photometrically and the intensity thereof depends on the chlorine content. As mentioned at the outset, the measurement is sensitive to interference by other disinfection components, other interfering ions, particles, etc.

Other reference measurements, such as the amperometric measurement or the turbidity content, can also be considered instead of or in addition to the photometric measurement.

According to the concept of the present disclosure, therefore, the provision 100 of a mobile calibration device 10, and in particular a modular calibration device, is first carried out.

FIG. 1 shows two variants for carrying out the method according to the present disclosure, wherein a first variant X is described first.

The provision 100 takes place at the measuring point 1. According to variant X, the provision 100 can take place by connecting 110 a supply line 5 to the measuring point 1, which allows for the transfer or supply 150 of calibration fluid between the calibration device 10 and the measuring point 1.

Furthermore, the provision 100 can optionally also comprise connecting 120 an outlet line 6 to the measuring point 1.

The start-up 200 of the calibration device 10 then takes place. For this purpose, the provision 210 of a stabilized calibration fluid 11 is made first.

The activation 220 of the calibration fluid 11 then takes place preferably by the addition of a reactant. The reactant preferably serves to change the pH, thereby releasing the species to be determined.

Finally, the activated calibration fluid is introduced into the measuring point 1 for calibrating the amperometric sensor 2. The calibration fluid can have a specific concentration of a species to be detected.

Previously, the process medium should ideally be emptied out of the measuring point 1. As a result, the reference measurement or calibration by the calibration fluid having the specific known concentration of the species to be detected cannot be disturbed by foreign ions. Calibration 300 of the sensor can thus be performed by a corresponding control device by comparing a target and actual value without removing the sensor from the measuring point.

Following the calibration process, the measuring point 1 is decoupled 400 from the calibration device 10, for example by disconnecting the supply line 5 and optionally also the outlet line 6.

In a second variant Y of the method according to the present disclosure, provision 100 of the calibration device 10 is likewise made at the measuring point 1, wherein the calibration device 10 is not connected to the measuring point 1, but is only positioned in the vicinity of the measuring point 1. Therefore, “at the measuring point” only means that the calibration process is carried out at the measuring point and not in the laboratory. Variant Y is also recommended in particular for heavily soiled sensor surfaces, which can be coated, for example, with deposits or a biofilm. Cleaning can take place before calibration here. The provision 100 can thus also comprise the removal of the amperometric sensor 2 from the holding device 3 and/or insertion into a receptacle in the calibration device.

Alternatively, the removal and/or the insertion of the amperometric sensor can take place after step 200—the initial start-up of the calibration device. In this step, a stabilized calibration fluid is again provided and activated by the addition of a reactant.

The activated calibration fluid can then be supplied to the amperometric sensor 2.

For this purpose, the calibration device 10 can have a storage tank 8 for the stabilized calibration fluid, a storage tank 9 for the reactant, a metering device 14 and a mixing and/or reaction tank 15. The reaction tank 15 is then fluidically connected to the holding device of the sensor 2 in the calibration device 10 and flows around a region that is sensed by the sensor with calibration fluid.

The amperometric sensor 2 then records the measurement data of the calibration fluid, which has a specific concentration of the species to be detected. Again, an actual and target value comparison can take place within the scope of the calibration process 300.

Finally, the calibrated sensor 2 is installed and/or positioned 400a in the holding device 3 of the measuring point 1. The holding device 3 can also be referred to as a process fitting.

In both variants, X and Y, an active calibration fluid is provided at the location of the measurement by a calibration device that allows the calibration fluid to be supplied to the amperometric sensor.

Optionally, the calibration device 10 can also have a flow-through fitting 16 having a sensor unit or a sensor element 16.1 in both variants of the method. This can comprise a photometer and a storage tank for a photometrically active reagent, for example DPD, so that, for example, for redundancy reasons or for testing the calibration fluid itself or in the case of unknown calibration fluids or insufficient activation, a photometric reference measurement can be used to calibrate the amperometric sensor.

The flow-through fitting 16 can also optionally have a holding device for receiving the amperometric sensor 2 when it has been removed.

As an alternative to the photometer, the flow-through fitting 16 can also comprise a pH sensor, a turbidity sensor and/or an amperometric sensor.

Particularly preferably, the flow-through fitting 16 and the amperometric sensor arranged therein are designed analogously to the process fitting 3 at the measuring point 1.

Due to the purity of the freshly activated calibration fluid, this reference measurement is only susceptible to interference to a very small extent.

The calibration device 10 can likewise optionally have a pH sensor 19 for monitoring the pH, in particular during the photometric reference measurement. However, a pH sensor is unnecessary if a pH buffer is used in the calibration fluid.

The calibration device can simultaneously be used as a cleaning device. For example, it can have a tank containing cleaning liquid that is introduced into the measuring point before or after the calibration process according to variant X.

As can be seen from FIGS. 2 and 3, the control unit that carries out the target/actual value comparison can also be a mobile computing unit, for example a mobile phone or a tablet or a laptop or the like.

By connecting the modular mobile calibration and cleaning unit to the measuring point, for example to fittings of the measuring point, users can calibrate their sensors directly with a defined calibration solution and eliminate influences due to cross sensitivity or interference factors in the process medium, for example water, waste water and the like.

The calibration can be carried out without the time delay that arises during analysis by means of a reference method and directly in the installation situation in the process of the customer and is thus less prone to errors.

In the case of heavy organic or inorganic loading of the process medium and corresponding contamination of the process fitting, it is possible to guide a cleaner through the fitting prior to calibration.

Furthermore, it is possible to design the calibration unit to have an additional fitting such that a sensor can be removed from the holding device of the measuring point and calibrated in situ, i.e., at the measuring point, in the mobile calibration device using defined calibration solutions and with the necessary inflow.

The calibration device can furthermore be designed to have a transmitter and optionally a battery supply so that it functions autonomously and can also be transported to and used at measuring points without additional power supply connection options.

There are currently no known calibration solutions for disinfectants, such as free chlorine or chlorine dioxide, or bromine. The present disclosure provides a calibration solution having a defined concentration in situ, i.e., at the measuring point, without requiring a reference measurement.

The design of the mobile calibration unit will be explained in more detail below.

In addition to the components described above, the mobile modular calibration unit 10 also has a pump 11 with which the activated calibration fluid is conveyed into the measuring point 1. The pump is connected to the mixing and reaction tank 15.

Accordingly, the calibration device 10 has a connection and/or valve block 24 that is downstream of the pump in terms of flow. The connection and/or valve block 24 has an outlet 26 to which the supply line 5 is connected. Optionally, an inlet can also be provided, by means of which the calibration fluid can be supplied the flow-through fitting 16 with the sensor elements for reference measurement after passing through the measuring point 1. As a result, any contaminants that may have been picked up at the measuring point are taken into account in the reference measurement.

In FIGS. 2 and 3, the storage tank 8 and 9 is realized as regions of a bottle 7 that are separated from one another. Because to a predetermined amount of disinfectant is typically mixed with a discrete amount of the reactant 33 for activation, the dimensions of the storage tanks 8, 9 in the bottle can ideally be coordinated in such a way that the two tanks are emptied and replaced at the same time. The bottle is fixed by a vessel support 7a, preferably in the upper case shell 13.

The storage tanks 8, 9 are connected to the metering device 14, which in FIGS. 2 and 3 is placed as a flat element on the mixing and reaction tank 15. The provision of the activated calibration fluid 32 takes place in the reaction tank 15.

A stirring unit, for example a magnet of a magnetic stirrer, can be arranged in the mixing and reaction tank 15 in order to homogenize the fluid.

Finally, the pump 11 is connected to the mixing and reaction tank 15.

For operating the pump, the metering unit, the stirring unit and the sensors of the flow-through fitting 16, the calibration device 10 can have a flat battery 21. Such batteries are sufficiently known, for example, from laptop computers. Optionally, a flat clamp system (not shown) can be provided adjacently to the battery 21 for power distribution to the corresponding components of the calibration device 10. Furthermore, a charging module 22 for the battery, which charging module has an interface 23 for connecting an external power supply, is arranged in the lower case shell 12. In the region of the interface 23, the case can have an opening so that a charging cable can be connected to the case from the outside, for example even in the closed state.

The components of the calibration device 10 described above are advantageously arranged in a case. FIGS. 2 and 3 show an exemplary arrangement of the components in a lower case shell 12 and an upper case shell 13.

As can also be seen from FIGS. 2 and 3, the flow-through fitting 16 having the sensor element(s) and the transmitters and cable connections 17 arranged thereon can have an overall height that, in the erected state, prevent the case from closing. The photometer is therefore held in a tiltable holder so that the photometer can be conveniently accommodated in the lower case shell when tilted by 90°.

The measurement of the amperometric sensor and/or the photometric reference measurement can be evaluated by a mobile terminal 27, for example a mobile phone. For this purpose, the mobile calibration device 10 has a holder 28.

If the mobile calibration device is also to be used as a cleaning device, the calibration device has a further storage tank 29 containing cleaning liquid. Said cleaning liquid can also be conveyed via the pump 11 and fed to the measuring point via the outlet 23 and the supply line 5.

Finally, in the embodiment as a case for position locking, the calibration device 10 can have for an open state of the case shell. This can be, for example, a lockable telescopic rod.

Furthermore, the flow-through fitting 16 can have a sensor element 16.1, preferably in the form of an amperometric sensor.

Overall, the calibration device thus comprises a set having a cleaning solution and a stable calibration solution and a reactant for activating the calibration solution, which is supplied in an inactive state and activated by the user.

Optionally, a waste container can also be provided that can be arranged next to the bottle with the storage tanks 8 and 9 in the case.

As an example in a low-load process (e.g. drinking water containing a mixture of chlorine/chlorine dioxide), the calibration device shown can be installed as follows. In this case, the process medium is process water.

First, the calibration device 10 is connected to the measuring point via the supply line 5. The measuring point 1 is blocked with respect to the process water and activated calibration solution 32 is sucked in and pumped through the fitting by means of the pump 11. The calibration solution can be discharged via the usual path; see the outlet line 6 and the gully connected thereto, or can be pumped in a circuit through an additional connection at the armature outlet. For reasons of redundancy, guiding said solution in a circuit can be used for the amperometric sensor for the photometric reference measurement. It is not absolutely necessary if the concentration of the species to be detected in the calibration fluid is predefined and therefore known. The calibration solution thereby stabilized for transport and storage and is activated by the operator during calibration.

An example of a calibration fluid is indicated below.

The activated calibration fluid 32 is finally composed of a chlorine solution with a specific predefined concentration, for example 1 mg/L at a defined pH, for example, pH 7.

The following can be used as the stabilized calibration fluid 31:

### Variant A:

10% NaOCl at pH>12, with stabilizer, e.g. sodium pentaphosphate
pH buffer, e.g. pH 7 for activation as a pH-changing reactant

### Variant B:

solution with 1 mg/L of NaOCl at pH>12, with stabilizer, e.g. sodium pentaphosphate
salt mixture for pH buffer for activation as a pH-changing reactant

As described above, the calibration device can be used to calibrate amperometric sensors, in particular amperometric disinfection sensors, inside or outside the process installation situation using a defined stabilized calibration fluid. The calibration solutions are prepared or activated in situ from a plurality of components.

The calibration can take place without a photometric reference measurement. The calibration unit can be operated autonomously and transported to different measuring points.

A preferred use of the calibration method according to the present disclosure is for the calibration of disinfection sensors, preferably in water treatment, in particular in drinking water treatment.

Claimed is:

1. A method for calibrating an amperometric sensor at a stationary measuring point, comprising:
A: providing a mobile calibration device including a stabilized calibration fluid at the stationary measuring point;
B: activating the stabilized calibration fluid and providing an activated calibration fluid; and
C: calibrating the amperometric sensor using the activated calibration fluid.

2. The method according to claim 1, wherein the amperometric sensor is fixed at the measuring point in a process line or a process container when method steps A-C are carried out.

3. The method according to claim 1, wherein the amperometric sensor is provided in a holding device of the calibration device when method step C is carried out.

4. The method according to claim 1, wherein the activated calibration fluid has a disinfectant.

5. The method according to claim 1, wherein, after step B, the activated calibration fluid is supplied to the measuring point.

6. The method according to claim 1, wherein the activation process is performed by addition of a pH-changing reactant.

7. The method according to claim 1, wherein the activation process and the calibration process take place within less than 10 minutes.

8. The method according to claim 1, further comprising:
supplying cleaning liquid to the measuring point, at least before the calibration process in step C.

9. The method according to claim 1, further comprising:
recording an actual value via the amperometric sensor and comparing a target/actual value, wherein the target value is determined from a predefined concentration of a compound in the stabilized calibration fluid, which is converted into a species detectable by the amperometric sensor during the activation process in step B.

10. The method according to claim 3, wherein, after the calibration process, the amperometric sensor is reinserted into the measuring point, or in that, after the calibration process, the at least one fluid line or the plurality of fluid lines between the calibration device and the measuring point is/are dismantled.

11. The method according to claim 1, wherein the calibration process in step C is carried out as a measurement of the calibration fluid by the amperometric sensor and as a measurement of the calibration fluid by a reference measurement, a reference turbidity content measurement, an amperometric reference measurement, and/or a pH measurement.

12. A mobile calibration device for calibrating an amperometric sensor at a stationary measuring point, comprising:
a storage tank containing a stabilized calibration fluid;
a pump for transporting the calibration fluid to the measuring point or to a flow-through fitting having an internal holding device for the amperometric sensor; and
a case-shaped housing,
wherein the calibration device is designed as a portable device.

13. The mobile calibration device according to claim 12, further comprising:
a storage container for a reactant for activating the calibration fluid; and
a metering device for metering the reactant into the storage tank for the stabilized calibration fluid or into a mixing and reaction tank.

14. The mobile calibration device according to claim 13, further comprising:
an integrated power supply.

15. The mobile calibration device according to claim 12, further comprising:
a flow-through fitting having one or more sensor elements for redundancy measurement.

16. The method according to claim 4, wherein the disinfectant is a halogen compound, an oxidizing agent, free chlorine, chlorine dioxide, free bromine, or hydrogen peroxide.
